(19) United States
(12) Patent Application Publication
(10) Pub. No.: US 2014/0180110 A1
(43) Pub. Date: Jun. 26, 2014

(54) System and Method for Wireless Ultrasound Probe Pairing

(71) Applicant: GENERAL ELECTRIC COMPANY, Schenectady, NY (US)

(72) Inventor: Leif Schmedling, Toensberg (NO)

(73) Assignee: GENERAL ELECTRIC COMPANY, Schenectady, NY (US)

(21) Appl. No.: 13/721,270

(22) Filed: Dec. 20, 2012

(51) Int. Cl. A61B 8/00; A61B 8/14

(52) U.S. Cl. CPC A61B 8/4472; A61B 8/145; A61B 8/4438
USPC 600/447

(57) ABSTRACT

A system for pairing a wireless ultrasound probe and an ultrasound scanner is provided. The ultrasound system comprises an ultrasound scanner comprising a near field communication reader capable of generating a near field communication activation field and a wireless ultrasound probe comprising a near field communication device, wherein the probe is adapted to transmit pairing information to the scanner via a near field communication protocol.

### BACKGROUND OF THE INVENTION

In the near future the use of wireless ultrasound probes in ultrasonography will be widespread and wireless echo will quickly become the de facto standard. This will become reality when wireless technologies mature and are able to meet the requirements to bandwidth and power consumption set by ultrasound equipment. Several wireless technologies can, at the current time, be considered viable paths; examples of such technologies are Bluetooth® and Wi-Fi.

In order for the ultrasound scanner to be able to communicate and operate a wireless ultrasound probe, the scanner, the probe, or both the scanner and probe, must be configured to recognize each other. Traditionally where wireless technologies have been applied this has been done by manually configuring the client side (in this case the scanner) with address, protocol, speed and other details required to reach the server (in this case the wireless probe). This configuration process is usually cumbersome and prone to errors. Additionally, the process is unduly repetitive as the configuration would have to be performed for each probe added for use with the system and would also have to be repeated whenever a probe is moved to a different scanner. As the configuration process is troublesome, it becomes less desirable to transfer probes between scanners, resulting in typically a one probe per scanner set up.

Current concerns with the use of wireless probes include power consumption. Current wireless technologies such as Bluetooth and Wi-Fi are high bandwidth and have proportionally high power consumption rates. This can rapidly drain the battery life of the wireless probe. If a probe were to run out of battery life before the ultrasonography procedure is complete, the sonographer would need to configure a different probe, most likely slowing down workflow and throughput.

For these and other reasons an easier and less error prone method for pairing wireless ultrasound probes to ultrasound scanners is desired.

### BRIEF DESCRIPTION OF THE INVENTION

The above-mentioned shortcomings, disadvantages and problems are addressed herein which will be understood by reading and understanding the following specification.

In an embodiment, an ultrasound system is provided that includes an ultrasound scanner having a near field communication reader capable of generating a near field communication activation field. The system further includes a wireless ultrasound probe having a near field communication device, wherein the probe is adapted to transmit pairing information to the scanner via a near field communication protocol.

In an embodiment, an ultrasound system is provided that includes an ultrasound scanner having a near field communication reader that defines a near field communication activation field. The system further includes a wireless ultrasound probe having a near field communication device that creates a communication area, wherein the probe is adapted to transmit pairing information to the scanner via a near field communication protocol when the probe is brought within range of the near field communication activation field.

In another embodiment, a method of pairing a wireless probe to an ultrasound scanner comprises positioning a near field communication enabled wireless ultrasound probe in range of a near field communication enabled ultrasound scanner. When the probe and scanner are in range, a near field communication link is established between the probe and the scanner. The probe and scanner transmit pairing information between the probe and the scanner such that the probe is paired to the scanner.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment of the disclosure;

FIG. 2 is a simplified block diagram of an ultrasound probe formed in accordance with an embodiment of the disclosure;

FIG. 3 is a perspective view of an ultrasound imaging system in accordance with an embodiment of the disclosure; and

FIG. 4 is a flow chart illustrating a method in accordance with an exemplary embodiment of the disclosure.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

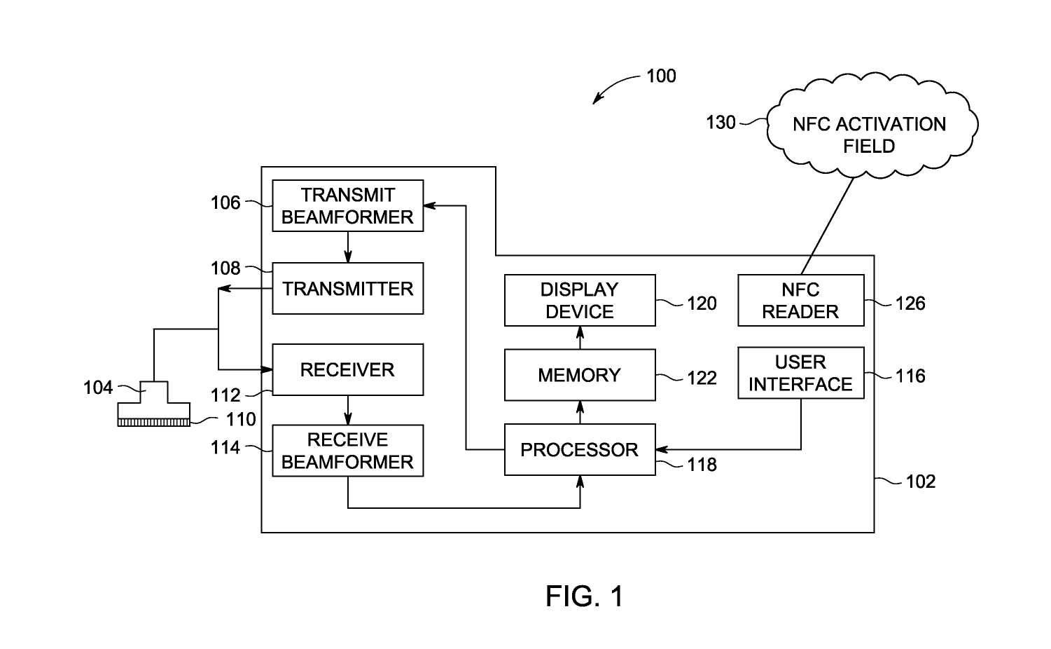

FIG. 1 is a schematic diagram of an ultrasound imaging system 100 in accordance with an embodiment of the disclosure. The ultrasound imaging system 100 comprises ultrasound scanner 102 and probe 104. The ultrasound scanner 102 may include a transmit beamformer 106 and a transmitter 108 that drive elements 110 within the probe 104 to emit pulsed ultrasonic signals into a body (not shown). The probe 104 may be an 2D array probe according to an embodiment. However, any other type of probe that is fully steerable in an elevation direction and capable of acquiring four-dimensional (4D) ultrasound data may be used according to other embodiments. For purposes of this disclosure, the term four-dimensional ultrasound data, or 4D ultrasound data, is defined to include ultrasound data including multiple volumes of a region-of-interest acquired over a period of time. The 4D ultrasound data contains information about how a volume changes over time. Each of the volumes may include a plurality of 2D images or slices.

Still referring to FIG. 1, the pulsed ultrasonic signals are back-scattered from structures in the body, such as blood cells or muscular tissue, to produce echoes that return to the elements 110. The echoes are converted into electrical signals, or ultrasound data, by the elements 110 and the electrical signals are transmitted wirelessly to the scanner 102 and received by a receiver 112. The electrical signals representing the received echoes are passed through a receive beamformer 114 that outputs ultrasound data. According to some embodiments, the probe 104 may contain electronic circuitry to do all or part of the transmit and/or the receive beamforming. For example, all or part of the transmit beamformer 106, the transmitter 108, the receiver 112 and the receive beamformer 114 may be situated within the probe 104. The terms “scan” or “scanning” may also be used in this disclosure to refer to acquiring data through the process of transmitting and receiving ultrasonic signals. The terms “data” or “ultrasound data” may be used in this disclosure to refer to either one or more datasets acquired with an ultrasound imaging system. A user interface 116 may be used to control operation of the ultrasound imaging system 100, including, to control the input of patient data, to change a scanning or display parameter, and the like.

The ultrasound imaging system 100 also includes a processor 118 to control the transmit beamformer 106, the transmitter 108, the receiver 112 and the receive beamformer 114. The processor 118 is in wireless communication with the probe 104. The processor 118 may control the probe 104 to acquire data. The processor 118 controls which of the elements 110 are active and the shape of a beam emitted from the probe 104. The processor 118 is also in electronic communication with a display device 120, and the processor 118 may process the data into images for display on the display device 120. For purposes of this disclosure, the term “electronic communication” may be defined to include both wired and wireless connections.

The processor 118 may include a central processor (CPU) according to an embodiment. According to other embodiments, the processor 118 may include other electronic components capable of carrying out processing functions, such as a digital signal processor, a field-programmable gate array (FPGA) or a graphic board. According to other embodiments, the processor 118 may include multiple electronic components capable of carrying out processing functions. For example, the processor 118 may include two or more electronic components selected from a list of electronic components including: a central processor, a digital signal processor, a field-programmable gate array, and a graphic board. According to another embodiment, the processor 118 may also include a complex demodulator (not shown) that demodulates the RF data and generates raw data. In another embodiment the demodulation can be carried out earlier in the processing chain.

The processor 118 may be adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. For example, an embodiment may acquire and display data a real-time volume-rate of 7-20 volumes/sec. However, it should be understood that the real-time frame rate may be dependent on the length of time that it takes to acquire each volume of data. Accordingly, when acquiring a relatively large volume of data, the real-time volume-rate may be slower. Thus, some embodiments may have real-time volume-rates that are considerably faster than 20 volumes/sec while other embodiments may have real-time volume-rates slower than 7 volumes/sec. The data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a volume-rate of, for example, 10 Hz to 30 Hz. Images generated from the data may be refreshed at a similar volume-rate. Other embodiments may acquire and display data at different rates. For example, some embodiments may acquire data at a volume-rate of less than 10 Hz or greater than 30 Hz depending on the size of the volume and the intended application. A memory 122 is included for storing processed frames of acquired data. In an exemplary embodiment, the memory 122 is of sufficient capacity to store at least several seconds worth of frames of ultrasound data. The frames of data are stored in a manner to facilitate retrieval thereof according to its order or time of acquisition. The memory 122 may comprise any known data storage medium.

In various embodiments of the present invention, data may be processed by other or different mode-related modules by the processor 118 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from coordinates beam space to display space coordinates. A video processor module may be provided that reads the image frames from a memory and displays the image frames in real time while a procedure is being carried out on a patient. A video processor module may store the image frames in an image memory, from which the images are read and displayed.

The ultrasound scanner 102 also comprises a near field communication (hereinafter abbreviated “NFC”) reader 126. NFC is a short range wireless communication protocol that has been standardized in ISO/IEC 18092/ECMA-340 and ISO/IEC 21481/ECMA-352. As understood by those skilled in the art, the NFC reader 126 is adapted to emit a small electric current, which creates a magnetic field that defines a NFC activation field 130. The NFC activation field extends up to a distance of about 20 cm from the NFC reader 126. It should be appreciated, however that the NFC activation field may extend less than 20 cm. For example, in one embodiment, the NFC activation field extends about 10 cm, while in another embodiment the NFC activation field extends 4 cm. It should also be appreciated that the NFC activation field may extend to more than 20 cm so long as the field can be generated by a NFC protocol.

The NFC reader 126 may be adapted to read information, send information, or both. As such, the NFC reader 126 may be capable of receiving the magnetic field from another NFC-enabled device to communicate data or other information.

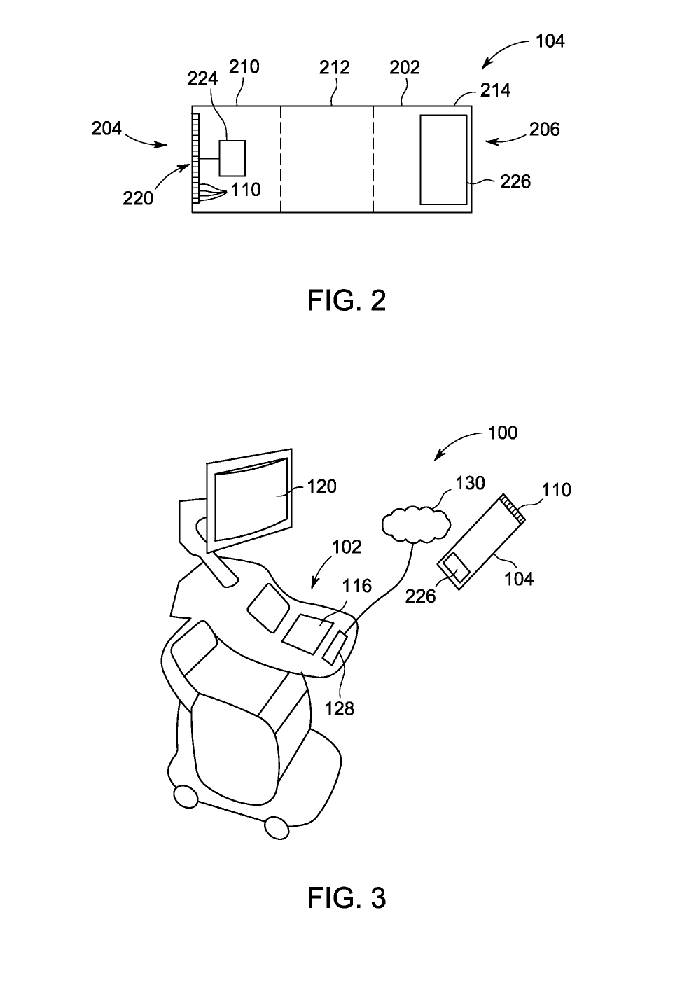

FIG. 2 illustrates a block diagram of an exemplary ultrasound probe 104 that is formed in accordance with an exemplary embodiment. The ultrasound probe 104 is configured to be held in the palm of an operator's hand. The ultrasound probe 104 generally includes a housing 202 having a proximal end 204 and a distal end 206. The housing 202 includes a proximal portion 210, a connecting portion 212 and a distal portion 214. The proximal portion 210 is disposed proximate to the proximal end 204. The distal portion 214 is disposed proximate to the distal end 206. The connecting portion 212 is disposed between the proximal portion 210 and the distal portion 214.

The proximal portion 210 generally includes therein control components and operating components for performing ultrasound scans. For example, and in general, the proximal portion 210 may include therein a transducer array 220 that is located at the proximal end 204 of the housing 202. The transducer array 220 may include a plurality of elements 110, for example piezoelectric elements, and control components 224, for example, electrical components mounted to a printed circuit board (not shown). The proximal portion 210 may be used to scan a patient by emitting therefrom ultrasonic waves and receiving echoes which are utilized to reconstruct an image of the area being scanned. It should be noted that the ultrasound probe 104 may include additional component parts, for example, a control knob (not shown) that is used to control the operation of the ultrasound probe.

The connecting portion 212 couples the proximal portion 210 to the distal portion 214. Specifically, the connecting portion 212 provides a mating interface between the proximal portion 210 and the distal portion 214 to enable the proximal portion 210 to be physically coupled to the distal portion 214. In the exemplary embodiment, the distal portion 214 includes a NFC communication device 226 that is located proximate to the distal end 206. NCF communication device 226 may be a NFC reader, adapted to read and send information. However, it should be appreciated that the NFC communication device may alternately be a NFC tag, be adapted to only send information. The NFC communication device creates a near field communication area around the probe. The NFC communication device is able to communicate with other devices when the devices are in the communication area.

NFC communication device 226 contained in the probe may be adapted to send pairing information. Pairing information may comprise at least one of primary communication information and probe identification information. Primary communication information may include wireless protocol, address, baud rate, encryption key, channel or any combination thereof. The wireless protocol preferably has a bandwidth higher than that of NFC. In the exemplary embodiment wireless protocol may be Bluetooth or Wi-Fi, but other wireless protocols may be envisioned. Probe identification information may include probe name, serial number, scanning capabilities, calibration data, manufacturer, manufacture date, battery level, estimated battery life, self-diagnosis or any combination thereof.

Still referring to FIG. 2, the ultrasound probe 104, in one embodiment, is configured to generate ultrasound data based on the echoes and to wirelessly transmit the ultrasound data to a remote device that is configured to reconstruct an image based on the received data. Optionally, the ultrasound probe 104 may be wired to the ultrasound scanner to transmit the ultrasound data to the scanner for image reconstruction.

The probe 104 functions in at least a sleep state and an active state. In the sleep state, some of the internal subsystems of the probe 104 are powered and thus functional while others are powered down. For example, all subsystems are powered down, except for the NFC subsystem. In the sleep state, the probe 104 is able to conserve battery power. In the active state, all subsystems of the probe 104 are powered.

The probe 104 can transition from the sleep state to the active state in at least three ways. First, the probe 104 may contain an accelerometer (not pictured) or some other sensor enabled to sense movement. Upon sensing movement, the probe would transition from the sleep state to the active state. Alternatively, the probe 104 may comprise a button or user interface (not pictured) that the user would engage to transition the probe from the sleep state to the active state. Still alternatively, the probe 104 may simply be introduced or placed within the NFC activation field created near the ultrasound scanner 102, such as illustrated by reference number 130 in FIG. 1. When the probe is positioned within the activation field, the probe 104 transitions from the sleep state to the active state.

FIG. 3 will now be described in accordance with an exemplary embodiment. The ultrasound system comprises an ultrasound scanner 102 and a wireless ultrasound probe 104. The ultrasound scanner may comprise a display device 120, a user interface 116 and a NFC activation field 130. The NFC activation field extends up to a distance of about 20 cm from the NFC reader that is concealed beneath the outer housing of the scanner in the area visually indicated to the user by the outline 128 in FIG. 3. The outlined area 128 provides indicates the area of the activation field for an operator to place the probe during the pairing process. It should be appreciated, however that the NFC activation field may extend less than 20 cm. For example, in one embodiment, the NFC activation field extends about 10 cm, while in another embodiment the NFC activation field extends 4 cm. It should also be appreciated that the NFC activation field may extend to more than 20 cm so long as the field can be generated by a NFC protocol.

The probe 104 comprises elements 110 and NFC communication device 226. To pair the wireless probe 104 with the scanner 102, a user may simply bring the NFC communication device 226 of probe 104 into the NFC activation field 130 of the scanner. The probe 104 may be passed through the NFC activation field 130 or the probe may be held or placed in the NFC activation field 130. Both actions enable the probe 104 and scanner 102 to communicate via NFC.

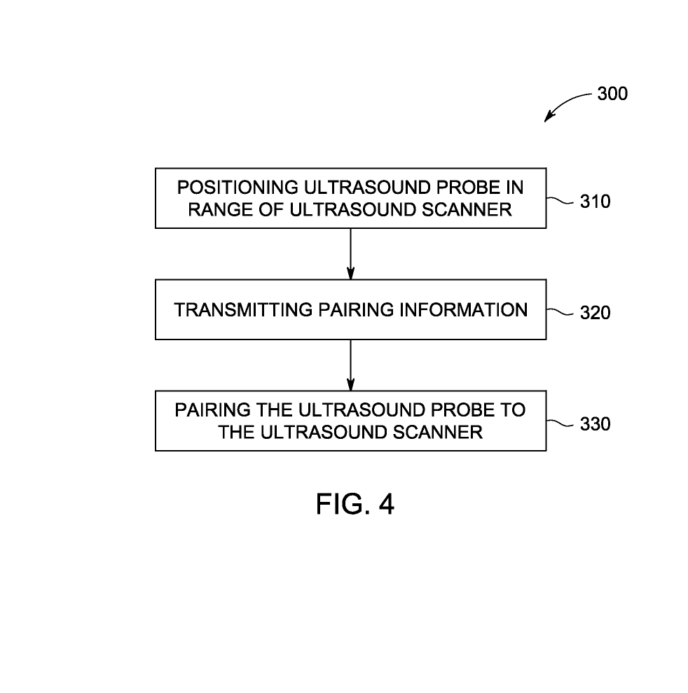

An exemplary method for pairing a wireless probe to an ultrasound scanner is generally depicted in FIG. 4 in accordance with one embodiment. The exemplary method 300 may include positioning a NFC enabled wireless ultrasound probe in range of a NFC enabled ultrasound scanner wherein a NFC link is established between the probe 104 and the scanner 102 in step 310. The range extends up to a distance of about 20 cm. It should be appreciated, however that the range may extend less than 20 cm. For example, in one embodiment, the range extends about 10 cm, while in another embodiment the range extends 4 cm. It should also be appreciated that the NFC activation field may extend to more than 20 cm so long as the field can be generated by a NFC protocol.

Step 320 may comprise transmitting pairing information between the ultrasound probe 104 and the ultrasound scanner 102. Pairing information may comprise at least one of primary communication information and probe identification information. Primary communication information comprises wireless protocol, address, baud rate, encryption key and channel. The wireless protocol may have a bandwidth higher than that of NFC. Bluetooth and Wi-Fi are examples of such wireless protocols, however it should be appreciated that other wireless protocols may be envisioned Probe identification information comprises name, serial number, scanning capabilities, calibration data, manufacturer, manufacture date, battery level, estimated battery life, and self-diagnosis.

In one embodiment, primary communication information is transmitted. For example, the wireless protocol information is sent from the probe to the scanner. In another embodiment, probe identification information may be transmitted. In this embodiment, the scanner may comprise a library that contains a database of primary communication information based on the probe identification information. Therefore, for example, when a probe transmits its name or serial number, the scanner is able to match the identification information with the respective primary communication information.

Step 330 of method 300 may include pairing the ultrasound probe 104 to the scanner 102. Once pairing information is transmitted, the scanner 102 and probe 104 are able to communicate via a primary wireless protocol. The primary wireless protocol may be a higher bandwidth wireless protocol than NFC, such as Bluetooth or Wi-fi, which is capable of accommodating higher bandwidth activities such as scanning. It should be appreciated, however, that Bluetooth and Wi-Fi are example wireless protocols and other wireless protocols could be envisioned.

An ultrasound system comprising NFC technology can immensely improve ultrasonography workflow and system usability. While NFC does not provide the bandwidth required for scanning, it is ideal for transmitting pairing information at a low power consumption bandwidth allowing the scanner and probe to connect via a higher bandwidth wireless technology for scanning.

The NFC enabled ultrasound system as described in the exemplary embodiments provides several commercial advantages. First, the use of NFC provides a seamless user experience. As probe configuration is not needed, the sonographer can easily switch between different probes simply by selecting a probe and swiping it over NFC activation field. The sonographer could be ready to start scanning within seconds. There is no need to press any buttons or manually enter information into the scanner.

Another commercial advantage is how easy it is to move NFC enabled probes between NFC enabled scanners. This would be attractive to customers as there would be no need for purchasing a complete set of probes for each scanner. The customer could expand their probe inventory with new probes as they see fit over time, allowing customers to strategically plan purchases and possibly save money. NFC enabled probes and scanners would also be capable of accommodating the use of probes and scanners manufactured by different manufacturers. Additionally, ultrasound system installation at the customer site would be simplified, potentially cutting manufacturing and service cost for the supplier and cutting administration costs for the customer.

Enabling probes and ultrasound scanners with NFC technology is an inexpensive solution. Various configurations of NFC technology are envisioned. For example, mid-end to low-end probes may comprise a NFC tag that is only able to send information. The NFC tag would not require that the probe be powered—instead it can be powered by inducing a communication between itself and a NFC reader, in this case, comprised in the scanner. Thus NFC in the probes can be realized as easily as by applying a preprogrammed NFC tag, such as a sticker, on a probe. NFC tags are readily available with cost in the range of 1 USD. On the other hand, high-end products may comprise NFC readers in both probe and the scanner for two-way communication in order to provide more features.

Another important feature of a wireless probe is battery life. NFC can be used to save battery life, potentially extending the scanning time of the probe. As described herein, NFC accommodates the probe having a sleep state and an active state wherein upon bringing the probe in proximity to the activation field on the scanner the probe would wake up from sleep and enter the active state where all subsystems of the probe are powered. Also, transmitting probe identification information, such as battery life, during the pairing process would allow the sonographer to select an appropriate probe for a procedure. The sonographer could also immediately be notified of the estimated battery time left on the probe upon simply by bringing it in proximity to the scanner potentially saving the sonographer for headaches due to low battery during an exam.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. An ultrasound system comprising:
an ultrasound scanner comprising a near field communication reader capable of generating a near field communication activation field; and
a wireless ultrasound probe comprising a communication device that creates a near field communication area, wherein the probe is adapted to transmit pairing information to the scanner via a near field communication protocol when the near field communication area overlaps the near field communication activation area.

2. The ultrasound system of claim 1, wherein the pairing information comprises probe identification information.

3. The ultrasound system of claim 2, wherein probe identification information comprises at least one of name, serial number, scanning capabilities, calibration data, manufacturer, manufacture date, battery level, estimated battery life, and self-diagnosis.

4. The ultrasound system of claim 1, wherein the pairing information comprises primary communication information.

5. The ultrasound system of claim 4, wherein the primary communication information comprises at least one of wireless protocol, address, baud rate, encryption key, and channel.

6. The ultrasound system of claim 4, wherein the primary communication information comprises a wireless protocol having a higher bandwidth than a bandwidth of the near field communication protocol.

7. The ultrasound system of claim 6, wherein the wireless protocol comprises at least one of Bluetooth and Wi-Fi.

8. The ultrasound system of claim 1, wherein the wireless probe comprises a sleep state and an active state, wherein the probe functions in the sleep state until the probe enters the activation field, wherein the probe then transitions to the active state.

9. The ultrasound system of claim 1, wherein the near field communication device is located on a distal end of the wireless ultrasound probe.

10. An ultrasound system comprising:
an ultrasound scanner comprising near field communication reader that creates a near field communication activation field, and
a wireless ultrasound probe comprising a near field communication device, wherein the probe is adapted to transmit pairing information to the scanner via a near field communication protocol when the probe is brought within range of the near field communication activation area.

11. The ultrasound system of claim 10, wherein the range of the near field communication activation area is about 20 cm or less.

12. The ultrasound system of claim 11, wherein the pairing information comprises probe identification information and primary communication information.

13. The ultrasound system of claim 12, wherein the pairing information comprises at least one of wireless protocol, address, baud rate, encryption key, and channel, and the probe, identification information comprises at least one of name, serial number, scanning capabilities, calibration data, manufacturer, manufacture date, battery level, estimated battery life, and self-diagnosis.

14. A method of pairing a wireless probe to an ultrasound scanner comprising:
positioning a near field communication enabled wireless ultrasound probe in communication range of a near field communication enabled ultrasound scanner;
establishing a near field communication link between the wireless ultrasound probe and the scanner;
transmitting pairing information between the wireless ultrasound probe and the scanner; and
pairing the wireless ultrasound probe to the scanner.

15. The method of claim 14, wherein the pairing information comprises at least one of probe identification information and primary communication information.

16. The method of claim 14, wherein the pairing information is transmitted from the wireless ultrasound probe to the scanner.

17. The method of claim 14, further comprising the step of transitioning the wireless ultrasound probe from a sleep state to an active state prior to establishing the near field communication link.

18. The method of claim 17, wherein the transitioning to the active state occurs automatically upon movement of the wireless ultrasound probe.

19. The method of claim 14, wherein the range of the near field communication activation area about 20 cm or less.

20. The method of claim 14, wherein pairing information comprises at least one of probe identification information and primary connection information.

21. The method of claim 20, wherein probe identification information comprises at least one of name, serial number, scanning capabilities, calibration data, manufacturer, manufacture date, battery level, estimated battery life, and self-diagnosis.

22. The method of claim 20, wherein the primary communication information comprises at least one of wireless protocol, address, baud rate, encryption key, and channel.

23. The method of claim 14, wherein during transmission of the pairing information, the probe and scanner communicate via a primary wireless connection protocol.

24. The method of claim 23, wherein the primary wireless connection protocol is Wi-Fi.

25. The method of claim 24, wherein the primary wireless connection protocol is Bluetooth.
